(19) United States
(12) Patent Application Publication
(10) Pub. No.: US 2009/0114216 A1
(43) Pub. Date: May 7, 2009

(54) System And Method For Improving Breathing Pattern With Interactive Multi-Sensory Approach

(76) Inventors: Yi-Ping Hung, Taipei City (TW); King-Jen Chang, Taipei City (TW); Jin-Shing Chen, Taipei City (TW); Meng-Chieh Yu, Taipei City (TW)

Correspondence Address: LIN & ASSOCIATES INTELLECTUAL PROPERTY, INC., P.O. BOX 2339, SARATOGA, CA, 95070-0339, US

(21) Appl. No.: 12/032,726

(22) Filed: Feb. 18, 2008

(30) Foreign Application Priority Data
Nov. 6, 2007 (TW) 096141934

(51) Int. Cl. A61M 16/00

(52) U.S. Cl. 128/200.24; 600/534

(57) ABSTRACT

A system for improving a breathing pattern with an interactive multi-sensory approach is provided. The system includes a breathing condition detector and a processor, for guiding a user to learn a suitable breathing pattern. The breathing condition detector attached to the user, is adapted to detect a breathing condition according to a natural expansion or a natural shrinkage of a body of the user. The breathing condition at least includes a breathing mode (breast breathing or belly breathing), breathing rate, and breathing depth. The processor displays a relative breathing condition picture reflecting the detected breathing condition detected by the breathing condition detector on a monitor. As such, the user can understand the practical breathing condition by viewing the breathing condition picture, so as to be instructed to learn to use a breathing pattern suitable for him.

### BACKGROUND OF THE INVENTION

1. Field of the Invention

The present invention relates generally to a system and a method for improving a breathing pattern, and in particular, to a system and a method for improving a breathing pattern with an interactive multi-sensory approach.

2. The Prior Arts

Currently, office workers have to be used to take more responsibilities and bear heavier burden than ever before. They are often surrounded by negative feelings, e.g., anger, fear, nervousness, or the like, which are usually caused by overpressure. Such negative feelings may distort human's autonomic nervous system, so that one's breath may become fast-paced, short and hard to control, which often causes an insufficient oxygen content contained in the blood and harms human's health.

Generally speaking, ordinary body hypoxia even if does not directly threaten one's life, at least harm his health to some degree. Oxygen is as important for human beings as food and water do. Since any nourishment has to be first oxidized to release energy and thereafter the energy can be provided for the body. According to this concern, oxygen is critical for metabolism and probably one of the most important factors for maintaining human's health. If one stays in an environment short of oxygen supply, or either cannot inhale enough oxygen due to his body condition, or cannot effectively utilized inhaled oxygen although he is in an oxygen rich environment, changes of body function, metabolism, and configuration may occur. Typically, symptoms of anoxia include dizziness, headache, tinnitus, giddiness, limpness, and subsequent nausea, vomit, ecphysesis, and fast and weak heartbeat.

Regular metabolism starts from air inhaling and exchanging of the breathing system. Difficulty existed in any step of the whole breathing process will cause insufficient air exchange. In case an insufficient air exchange happens, body instantly takes action for compensatory protection. Driven by neural reflex, breath correspondingly become deeper and faster. When the compensatory protection cannot compensate the demand for oxygen, the anoxia occurs.

Anoxia, no matter for what reason it occurs, changes body's function and metabolism. Neural system is especially sensitive to anoxia, and even mild anoxia may cause malfunctions of one's brain and vision. Although brain is only about 2% to 3% of weight of the whole body, it consumes about 20% to 30% of oxygen consumed by the whole body, and is the organ which consumes most oxygen in the body, so that heart has to provide about 15% of blood to the brain. However, brain itself has almost no energy stored for backup, and all energy consumed thereby comes from fresh blood provided through blood vessel thereof. Oxygen is extracted from the fresh blood and provided for the brain to maintain the brain for performing regular physiological function. As such, brain is least sustainable organ again anoxia.

Heart is another organ that consumes a lot of oxygen, metabolizes fast, and contains little oxygen storage, and is also vulnerable and sensitive to anoxia. Serious anoxia may decrease a contraction force of cardiac muscle, slow the cardiac rate, and reduce blood supplied by the heart. These symptoms and anoxia often mutually aggravate one by another, and may even finally cause putrescence or aberrance of the cardiac muscle. Of course, chronic anoxia is more dangerous, and it may cause cardiac failure.

Learning to breath with a suitable breathing pattern not only helps to avoid the risk of anoxia or other related symptoms which hurt human bodies, but also is employed for curing emotional uncomfortableness, such as nervousness, awkwardness, anxiety, and heart throb. Generally, a suitable deep breathing pattern requires a slow and uniform breathing frequency, in which air is deeply inhaled into all of the lung, and then is all exhaled. The process of exhaling the air also helps to progressively relax the muscle. By alternatively tensing and relaxing main muscles of the body, one can experience different feelings of tension and relaxation respectively, so thus achieving a final object of relaxing the heart and the body and helping to tune up functions of all organs of the body.

People are different in physiological conditions, and there is no absolute criteria can be determined for a correct breathing pattern. However, there certainly would be a breaching pattern suitable for a certain people. Properly breathing is a special knowledge, and there are breathing pattern mentors teach people to properly breathe. Especially, professional instructions for suitable breathing pattern are more needed for those patients suffering serious breathing difficulties. However, an ordinary breath teaching system providing basic breathing guidance to guide ordinary breath learner to learn at anywhere and anytime would be very desirable. Accordingly, the present invention provides a system and a method for improving a breathing pattern with an interactive multi-sensory approach, which is convenient for ordinary users to learn suitable breathing pattern.

### SUMMARY OF THE INVENTION

A primary objective of the present invention is to provide a system and method for improving a breathing pattern with an interactive multi-sensory approach. According to the present invention, a breathing condition of a user is detected, and then data is fed back to the user with a multi-sensory approach. The feedback data, which can be easily understood, includes touch adapted for sensing vibration, temperature, or mild current, hearing adapted for sensing voice guidance, speech guidance, background voice, or affair sound effect, or vision for sensing ambient light, or multimedia content. According to the feedback data, the user can learn how to improve the breathing pattern, so as to further alleviate emotional uncomfortableness, such as nervousness, awkwardness, anxiety, and heart throb, and thus reform the body condition and psychological health. The present invention substantially eliminates the need for a professional clinical staff to explain complicated physiological data for the user to improve the breathing pattern.

For achieving the foregoing objective, the present invention provides a system and method for improving a breathing pattern with an interactive multi-sensory approach, incorporating with science and art including physiological sensing system and medical technology, multimedia interactive technology, smart shirt technology, and clinical assessment, for setting up a smart shirt system which is adapted for improving a personal breathing pattern and further improving the physical and psychological health.

According to an embodiment of the invention, a system for improving a breathing pattern with an interactive multi-sensory approach mainly includes a breathing condition detector and a processor, for guiding a user to learn a suitable breathing pattern. The breathing condition detector is attached to the user, and can adaptively detect the breathing condition according to a natural expansion or a natural shrinkage of a body of the user when breathing. The breathing condition at least includes a breathing mode (breast breathing or belly breathing), breathing rate, and breathing depth. The processor displays a relative breathing condition picture according to the detected breathing condition detected by the breathing condition detector on a monitor. As such, the user can understand the practical breathing condition by viewing the breathing condition picture, so as to be instructed to learn to use a breathing pattern suitable for him.

### BRIEF DESCRIPTION OF THE DRAWINGS

The present invention will be apparent to those skilled in the art by reading the following detailed description of a preferred embodiment thereof, with reference to the attached drawings, in which:

FIG. 1 is a schematic diagram of a smart shirt of a system for improving a breathing pattern with an interactive multi-sensory approach in accordance with the present invention;

FIG. 2 is a schematic diagram illustrating a system for improving a breathing pattern with an interactive multi-sensory approach in accordance with an embodiment of the present invention; and

FIG. 3 is a schematic diagram illustrating a system for improving a breathing pattern with an interactive multi-sensory approach in accordance with another embodiment of the present invention.

### DETAILED DESCRIPTION OF THE PREFERRED EMBODIMENT

The accompanying drawings are included to provide a further understanding of the invention, and are incorporated in and constitute a part of this specification. The drawings illustrate embodiments of the invention and, together with the description, serve to explain the principles of the invention.

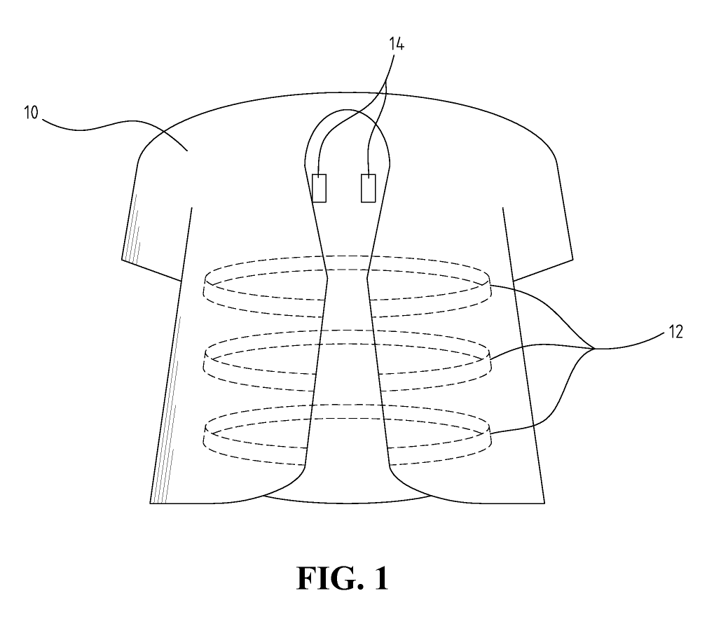

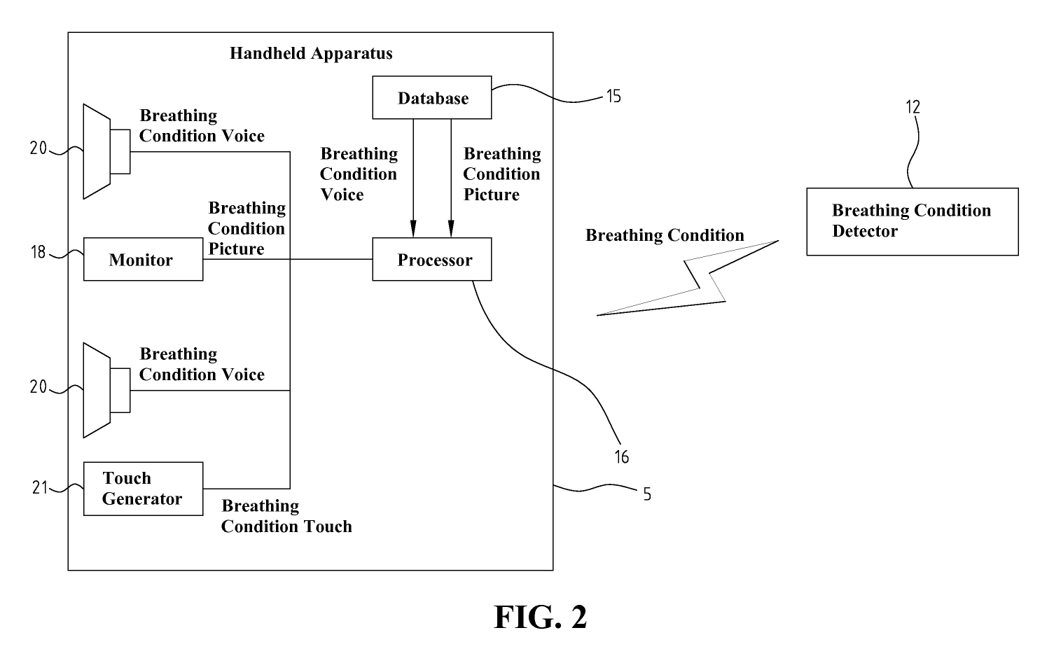

FIG. 1 is a schematic diagram of a smart shirt of a system for improving a breathing pattern with an interactive multi-sensory approach in accordance with the present invention. Referring to FIG. 1, there is shown a smart shirt 10 including at least one breathing condition detector 12 and a handheld apparatus 5 (as shown in FIG. 2). The handheld apparatus 5 is adapted to receive data transmitted from the breathing condition detector 12 by either a wireless connection or a wire connection. Specifically, according to an aspect of the invention, the smart shirt 10 includes three or more breathing condition detectors 12, at least parallel disposed therein at an upper side, a middle side, and a bottom side, respectively. The three or more breathing condition detectors 12 are adapted to synchronously detect breathing condition of a user. According to another aspect of the invention, in order to further improve precision of the detection, the smart shirt 10 may further include a physiological detector 14.

The physiological detector 14 is preferably disposed at a collar or near a collar of the smart shirt 10 to sense physiological data of the user at or near his neck. The physiological data include skin conductivity, blood pressure, blood oxygen concentration, body temperature, and electrocardiogram. Facilitating with the physiological detector 14, the user can also learn his physiological data variations when using the system for improving the breathing pattern with the interactive multi-sensory approach, so as to better understand the correlation between the breathing pattern and the physiological data. It should be noted that the smart shirt 10 is a preferred embodiment for exemplarily illustrating the system for improving the breathing pattern with the interactive multi-sensory approach only, and is not to limit the present invention. One skilled in the art may modify the present invention by employing other means to detect the breathing condition of a user within the scope of the present invention.

FIG. 2 is a schematic diagram illustrating a system for improving a breathing pattern with an interactive multi-sensory approach in accordance with an embodiment of the present invention. Referring to FIG. 2, there is shown a system for improving a breathing pattern with an interactive multi-sensory approach. The system includes a breathing condition detector 12, a multi-sensory feedback generator which can be a monitor 18, a speaker 20, or a touch generator 21, and a processor 16 adapted for controlling the multi-sensory feedback generators 18, 20 and/or 21 to transmit the breathing conditions. According to aspect of the embodiment, the system may optionally further includes a database 15. The multi-sensory feedback generators 18, 20, and/or 21 can provide multi-sensory feedback including touch, and/or hearing, and/or vision to the user to learn the breathing condition. The monitor 18 can be a head mounted display, a handheld monitor, or a PC monitor. The touch generator 21 can be a vibration motor, a thermal source, or a mild electricity power. Further, the thermal source may be a cold generator, while the mild electricity power may be a power controller.

Generally, the system for improving the breathing pattern with the interactive multi-sensory in accordance with the present invention employs a smart shirt 10 which uses the breathing condition detectors 12 to detect user's breathing condition and other physiological data, and uses the monitor 18, and/or the speaker 20, and/or the touch generator 21 to provide multimedia interactive feedbacks, so as to allow the user to learn a suitable breathing pattern and instruct the user to practice. The present invention provides multimedia displaying system of reflection, guidance, and assessment for the user by multi-sensory feedback including touch, hearing, and vision, so that the user can overcome negative emotions such as nervousness, awkwardness, anxiety, and heart throb, and further improve his physiological condition. This system eliminates the need for a professional staff to explain the complicated physiological data when learning the suitable breathing pattern.

When the user wears the smart shirt 10, the breathing condition detector 12 is attached to the user. The breathing condition detector 12 is adapted to detect a breathing condition according to a natural expansion or shrinkage of a body of the user. The breathing conditions include breathing mode, (breast breathing, belly breathing, underbelly breathing, or the like), breathing rate, and breathing depth. Then, the processor 16 receives the detected breathing conditions by either a wireless connection or a wire connection. The monitor 18 then displays a breathing condition picture corresponding to the breathing conditions. In such a way, the user can understand the breathing condition by viewing the breathing pattern, and be properly instructed to learn the suitable breathing pattern.

For example, in order to provide the user his breathing condition, different interactive touch feedback effects, hearing feedback effects and vision feedback effects can be selected according to the practical breathing condition. According to an aspect of the embodiment, touch feedback can be an option, for example a plurality of vibration motors can be equipped at different positions to adjust breathing frequency, breathing position, breathing depth of the user; cold/heat generator is equipped to reflect learning situation or physiological condition of the user; or a mild electricity is controlled to adjust breathing pace of the user. According to another aspect of the embodiment, hearing feedback is another option, for example a speaker can be used to synchronously play breathing guiding voice, a louder voice indicates more air should be inhaled, and a voice generating frequency for guiding the breathing frequency. According to a further aspect of the embodiment, vision feedback is a further option, for example more clouds indicate more suitable breathing pattern; and wading leaves of a tree indicate a less suitable breathing pattern. Facilitating with the foregoing multi-sensory feedback system, the user can more institutively learn to breathe in a relaxing way, so as to assist the user to learn in achieving a positive atmosphere.

The foregoing mentioned feedbacks should be happened corresponding to feasible, proper time for interactive sensing. As such, the present invention provides a systematic feedback mechanism of reflection, guidance, and assessment, for adapting appropriate sense feedback mode at proper time for strengthening the learning effect.

Reflection is to display the breathing condition and other physiological data by multimedia sense modes in time;

Guidance is to allow the user to feel the suitable breathing pace and follow the suitable breathing pace to learn the suitable breathing pattern.

Assessment is to provide an effect assessment, so as to allow the user to understand the learning effect.

Correlation of the multi-sensory including touch, hearing, and vision with reflection, guidance, and assessment is illustrated as below:

| | Reflection | Guidance | Assessment |
|---|---|---|---|
| Touch | Facilitating with | Guiding the user to | Different touch |
| | multi-sensory (touch) | properly practice | feeling are given to |
| | feedback generator to | breath with touching | the user depending |
| | instantly reflect breathing | sense of different | on whether the |
| | conditions of user (such as | frequencies, strengths | breathing condition |
| | employing a cold generator to | (such as vibration | is within an |
| | provide a cold feeling to | motors vibrating at | appropriate range |
| | the user when the user deep | different frequencies | |
| | breathes | or strengths) | |
| Hearing | Facilitating with | Guiding the user to | Different hearing |
| | multi-sensory (hearing) | properly practice | feeling are given to |
| | feedback generator to | breath with hearing | the user depending |
| | instantly reflect breathing | sense of different | on whether the |
| | conditions of the user (such | frequencies, strengths | breathing condition |
| | as employing a simulated | (such as playing a | is within an |
| | voice of wind to reflect | simulated voice of | appropriate range |
| | breathing volume of the | ocean wave, according to | |
| | user, in which a louder | which the user can | |
| | voice of wind indicates a | control his own | |
| | larger breathing volume) | breathing frequency | |
| | | and depth) | |
| Vision | Facilitating with | Guiding the user to | Different vision |
| | multi-sensory (vision) | properly practice | feeling are given to |
| | feedback generator to | breath with different | the user depending |
| | instantly reflect breathing | vision sense (such as | on whether the |
| | conditions of the user (such | introducing a | breathing condition |
| | as employing a tree with | simulated fog of | is within an |
| | leaves in which quantity | different density to | appropriate range |
| | and color are used to | guide the user to | (such as more |
| | indicate breathing volume | control his breathing | flourish and greener |
| | of the user) | frequency and depth) | leaves indicate more |
| | | | suitable breathing |
| | | | pattern |

Regarding reflection, the processor 16 can control the touch generator 21 to provide a corresponding breathing condition touch; control the speaker 20 to play a corresponding breathing condition voice; and control the monitor 18 to display a corresponding breathing condition picture, so as to allow the user to understand the practical breathing condition. The foregoing touch generator 21 can be a vibration motor, a thermal source, or a mild electricity source.

Regarding guidance, comparing with a standard mode of breathing condition, the processor 16 is adapted to judge a degree of the practical breathing condition of the user deviating from the standard mode, and then control the touch generator 21 to provide a corresponding breathing condition touch; control the speaker 20 to play a corresponding breathing condition voice; and control the monitor 18 to display a corresponding breathing condition picture, so as to guide the user to learn the suitable breathing pattern. It should be noted that the aforementioned standard mode is based on a well-known concept that a deeper and slower breath is a better. However, different individuals may be suitable for different standards according to the differences of their physiological conditions. For example, one may be suitable for a breathing frequency (such as inhaling for 2 second, and exhaling for 6 seconds). In this manner, the system may determine the standard mode according to the physiological data (including height, weight, age, sex), and physiological condition (normal or recovered patient), as well as previous learning scores, and thus finding out a most suitable breathing pattern for learning.

Regarding assessment, the processor 16 assesses the breathing condition as proper or not depending on whether the breathing condition is within a standard range or not. Then corresponding data is retrieved from a touch database 15 and performs a breathing condition touch with the touch generator 21, plays a breathing condition voice with the speaker 20, or displays a corresponding breathing condition picture with the monitor 18 for the user.

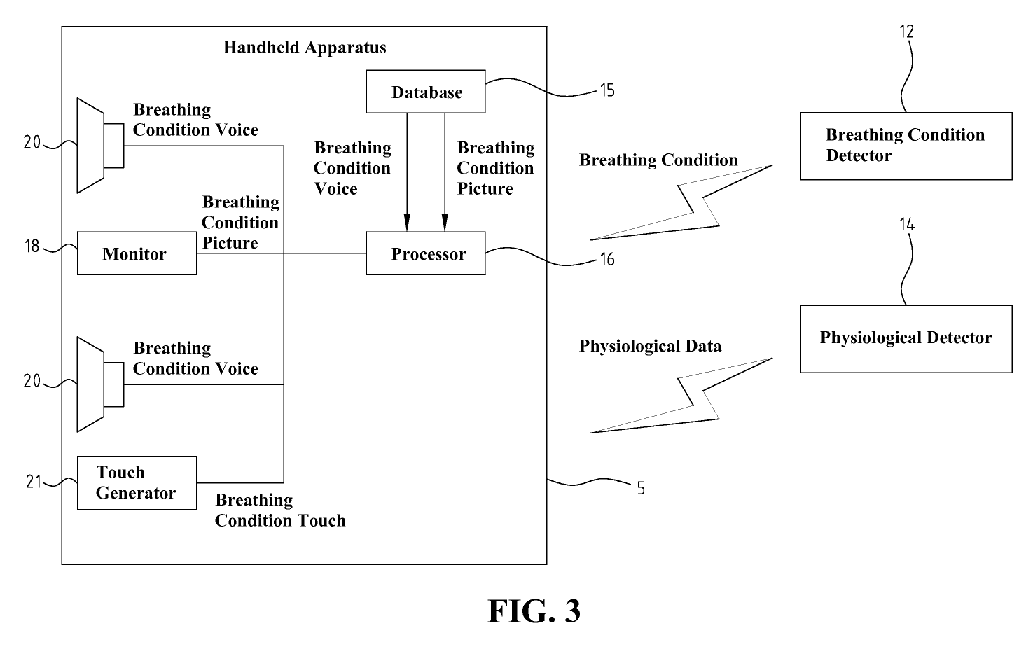

Referring to FIGS. 2 and 3, the system according to the present invention mainly includes the breathing condition detectors 12 adapted for detecting breathing conditions, the multi-sensory feedback generator which can be a monitor 18, a speaker 20, or a touch generator 21, and a processor 16 adapted for controlling the multi-sensory feedback generators 18, 20 and/or 21 to transmit the breathing conditions. The multi-sensory feedback generators can be adaptively selected according to the convenience and adaptability preferred by user, (for example, when driving, one should concentrate on the road condition, and therefore he may guide his breath by touch feeling) and thus providing different learning effects.

Different sense feelings of human beings have different characteristics. With respect to the convenience for sensing stimulation, the strengths thereof are ranked as touch, hearing, and vision in that order. As such, the touch generator 21 is a preferred one among the multi-sensory feedback generators. Affairs can be sensed by the touch generator 21 include vibration sense, cold/hot temperature sense, mild electricity sense. The speaker 20 can be used to sense affairs including voice guidance, speech guidance, background voice, or affair sound effect. The monitor 18 can be used to sense ambient light, or multimedia content. The affairs should happen at suitable times. Therefore, the present invention provides the systematic feedback mechanism of reflection, guidance, and assessment, which is to be further illustrated below.

In order to provide the convenience for the user to instantly sense the breathing condition, the database 15 stored in the handheld apparatus 5 is preferred to be a touch database including a plurality of breathing condition pattern touches, e.g., vibration. Each of the breathing condition pattern touches is further divided into a proper breathing pattern touch and an improper breathing pattern touch. In such a way, when the processor 16 receives a breathing condition identified as out of the standard range, a touch of an improper breathing condition pattern is retrieved from the touch database 15 and reflected by the vibration motor to allow the user to sense a one-time vibration. Similarly, when the processor 16 receives a breathing condition identified as within the standard range, a touch of a proper breathing condition pattern is retrieved from the touch database 15 and reflected by the vibration motor to allow the user to sense a three-time vibration.

In addition to the touch effect, voice effect can also be played when displaying the breathing condition picture. In this manner, the database 15 can also be a hearing database including a plurality of breathing condition voices. Each of the breathing condition voices is also further divided into a proper breathing pattern voice and an improper breathing pattern voice. In such a way, when the processor 16 receives a breathing condition identified as out of the standard range, a voice of an improper breathing condition pattern is retrieved from the hearing database 15 and reflected by the speaker 20 to play a corresponding breathing condition voice. Similarly, when the processor 16 receives a breathing condition identified as within the standard range, a voice of a proper breathing condition pattern is retrieved from the hearing database 15 and reflected by the speaker 20 to play a corresponding breathing condition voice.

Likewise, visional effect can also be played when displaying the breathing condition picture. In this manner, the database 15 can also be a vision database including a plurality of breathing condition pictures. Each of the breathing condition pictures includes a proper breathing pattern picture and an improper breathing pattern picture. In such a way, when the processor 16 receives a breathing condition identified as out of the standard range, a picture of an improper breathing condition picture is retrieved from the vision database 15 and reflected by the monitor 18 to display a corresponding breathing condition picture. Similarly, when the processor 16 receives a breathing condition identified as within the standard range, a picture of a proper breathing condition patter is retrieved from the vision database 15 and reflected by the monitor 18 to play a corresponding breathing condition picture. According to an aspect of the invention, the foregoing breathing condition pictures can be either a static picture or a dynamic picture.

It should be noted that the foregoing approaches, displaying pictures, playing voices, or vibration can be either individually used, or combined to produce a multimedia interactive game. In more details, when the user wears the smart shirt 10 and holds the handheld apparatus 5, he can play the multimedia interactive game of the system. The handheld apparatus 5 includes a touch pad (not shown hereby) by which the user is allowed to set the game and a menu thereof to enter the game in which he can conquer different barriers by practicing the skills about breathing mode, speed, and strength, and thus learning proper breathing pattern.

As for the system feedback part, it is preferred to provide in combination a PC large monitor to allow the user to view video of the game and a small screen on the handheld apparatus 5 to allow the user to control the menu and view simple cartoon video. According to an aspect of the present invention, other feedback parts, for example, the system voice or a hardware including light control, stepping motor control, may also be equipped to the system to provide multiple modes or multi-sensory multimedia interactive pattern for satisfying sensing nerves of the user.

Referring to FIG. 3, there is shown a system for improving a breathing pattern with an interactive multi-sensory approach according to another embodiment of the present invention. The system for improving a breathing pattern with an interactive multi-sensory approach includes a breathing condition detector 12, a physiological detector 14, a monitor 18, and a processor 16. According to an aspect of the embodiment, a speaker 20 or a database 15 can be optionally equipped thereto. The physiological detector 14 is attached to the body of the user similar to that shown in FIG. 1 and is adapted to detect physiological data of the user and provide the physiological data to the processor 16 for accurate judgment. The physiological data include skin conductivity, blood pressure, blood oxygen concentration, body temperature, and electrocardiogram.

Further, the breathing condition detected by the breathing condition detector 12 is mainly obtained in correspondence to figures of a relative standard range of suitable breast/belly breathing corresponding to users of different body forms provided by professional physicians. When more precision detection is required, physiological data detected by the physiological detector 14, e.g., skin conductivity, blood pressure, blood oxygen concentration, body temperature, and electrocardiogram, can also be referred in determining instant physiological feedback mechanism in further accordance with the relative standard range to remind or warning the user to improve and learn.

Comparing with the conventional approach of providing physiological data which displays only data and waveforms and requires a physician to explain, the present invention provides a system and a method for improving breathing pattern with an interactive multi-sensory approach, which introduces a multimedia feedback mechanism. The system employs a smart shirt to detect physiological data, and reflects the data with means of touch, hearing and vision, so as to instantly convert the physiological data and breathing learning data into feedback affairs which are convenient to understand, thus facilitating the learning of suitable breathing pattern by the systematic feedback mechanism of reflection, guidance, and assessment.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. A system for improving a breathing pattern with an interactive multi-sensory approach, comprising:
a breathing condition detector, attached to a user, being adapted to detect a breathing condition according to a natural expansion and a natural shrinkage of a body of the user when breathing, the breathing condition at least comprising a breathing mode, a breathing rate, and a breathing depth, and the breathing mode at least comprising breast breathing and belly breathing;
a multi-sensory feedback generator, providing a touch, and/or a hearing, and/or a vision sensing feedback; and
a processor, adapted for providing the detected breathing condition detected by the breathing condition detector to the user via the multi-sensory feedback generator.

2. The system according to claim 1, wherein the processor performs a corresponding breathing condition touch by a touch generator of the multi-sensory feedback generator, so as to allow the user to understand the breathing condition, the touch generator being a vibration motor, a thermal source, or a mild electricity source.

3. The system according to claim 1, wherein the processor plays a corresponding breathing condition voice by a speaker of the multi-sensory feedback generator, so as to allow the user to understand the breathing condition.

4. The system according to claim 1, wherein the processor displays a corresponding breathing condition picture by a monitor of the multi-sensory feedback generator, so as to allow the user to understand the breathing condition.

5. The system according to claim 1, wherein the processor is adapted to judge a degree of the breathing condition of the user deviating from a standard breathing condition, and then perform a corresponding breathing condition touch by a touch generator of the multi-sensory feedback generator, so as to guide the user to learn a suitable breathing pattern, the touch generator being a vibration motor, a thermal source, or a mild electricity source.

6. The system according to claim 1, wherein the processor is adapted to judge a degree of the breathing condition of the user deviating from a standard breathing condition, and then play a corresponding breathing condition voice by a speaker of the multi-sensory feedback generator, so as to guide the user to learn a suitable breathing pattern.

7. The system according to claim 1, wherein the processor is adapted to judge a degree of the breathing condition of the user deviating from a standard breathing condition, and then display a corresponding breathing condition picture by a monitor of the multi-sensory feedback generator, so as to guide the user to learn a suitable breathing pattern.

8. The system according to claim 1, further comprising:
a touch database, comprising a plurality of breathing condition pattern touches, each of which being divided into a proper breathing pattern touch and an improper breathing pattern touch,
wherein when the processor receives a breathing condition identified as out of a standard range, a touch of an improper breathing condition pattern is retrieved from the touch database and a touch generator of the multi-sensory feedback generator performs a corresponding breathing condition touch, the touch generator being a vibration motor, a thermal source, or a mild electricity source.

9. The system according to claim 1, further comprising:
a touch database, comprising a plurality of breathing condition pattern touches, each of which being divided into a proper breathing pattern touch and an improper breathing pattern touch,
wherein when the processor receives a breathing condition identified as within a standard range, a touch of a proper breathing condition pattern is retrieved from the touch database and a touch generator of the multi-sensory feedback generator performs a corresponding breathing condition touch, the touch generator being a vibration motor, a thermal source, or a mild electricity source.

10. The system according to claim 1, further comprising:
a hearing database, comprising a plurality of breathing condition voices, each of which being divided into a proper breathing pattern voice and an improper breathing pattern voice,
wherein when the processor receives a breathing condition identified as out of a standard range, a voice of an improper breathing condition pattern is retrieved from the hearing database and a speaker of the multi-sensory feedback generator plays a corresponding breathing condition voice.

11. The system according to claim 1, further comprising:
a hearing database, comprising a plurality of breathing condition voices, each of which being divided into a proper breathing pattern voice and an improper breathing pattern voice,
wherein when the processor receives a breathing condition identified as within a standard range, a voice of a proper breathing condition pattern is retrieved from the hearing database and a speaker of the multi-sensory feedback generator plays a corresponding breathing condition voice.

12. The system according to claim 1, further comprising:
a vision database, comprising a plurality of breathing condition pictures, each of which being divided into a proper breathing pattern picture and an improper breathing pattern picture,
wherein when the processor receives a breathing condition identified as out of a standard range, a picture of an improper breathing condition pattern is retrieved from the vision database and a monitor of the multi-sensory feedback generator displays a corresponding breathing condition picture.

13. The system according to claim 1, further comprising:
a vision database, comprising a plurality of breathing condition pictures, each of which being divided into a proper breathing pattern picture and an improper breathing pattern picture,
wherein when the processor receives a breathing condition identified as out of a standard range, a picture of a proper breathing condition pattern is retrieved from the vision database and a monitor of the multi-sensory feedback generator displays a corresponding breathing condition picture.

14. The system according to claim 12, wherein the breathing condition picture is a static picture or a dynamic picture.

15. The system according to claim 13, wherein the breathing condition picture is a static picture or a dynamic picture.

16. The system according to claim 12, wherein the monitor is a projector, a head mounted display, a PC display, a liquid crystal display, or a screen of a handheld apparatus.

17. The system according to claim 13, wherein the monitor is a projector, a head mounted display, a PC display, a liquid crystal display, or a screen of a handheld apparatus.

18. The system according to claim 1, further comprising:
a physiological detector, attached to the user for detecting a physiological data for provision to the processor in facilitating to make more precision judgment according to the breathing condition.

19. A method for improving a breathing pattern with an interactive multi-sensory approach, comprising:
detecting a breathing condition of a user according to a natural expansion and a natural shrinkage of a body of the user; and
feeding back the detected breathing condition to the user via a touch, and/or a hearing, and/or a vision sense,
wherein the breathing condition at least comprises a breathing mode, a breathing rate, and a breathing depth, and the breathing mode at least comprises breast breathing and belly breathing.

20. The method according to claim 19, wherein a touch generator is employed to perform a corresponding breathing condition touch so as to allow the user to understand the breathing condition, the touch generator being a vibration motor, a thermal source, or a mild electricity source.

21. The method according to claim 19, wherein a speaker is employed to play a corresponding breathing condition voice so as to allow the user to understand the breathing condition.

22. The method according to claim 19, wherein a monitor is employed to display a corresponding breathing condition picture so as to allow the user to understand the breathing condition.

23. The method according to claim 19, wherein a degree of the breathing condition of the user deviating from a standard breathing condition is detected, and a touch generator generates a corresponding breathing condition touch to guide the user to learn a suitable breathing pattern, the touch generator being a vibration motor, a thermal source, or a mild electricity source.

24. The method according to claim 19, wherein a degree of the breathing condition of the user deviating from a standard breathing condition is detected, and a speaker plays a corresponding breathing condition voice to guide the user to learn a suitable breathing pattern.

25. The method according to claim 19, wherein a degree of the breathing condition of the user deviating from a standard breathing condition is detected, and a monitor displays a corresponding breathing condition picture to guide the user to learn a suitable breathing pattern.

26. The method according to claim 19, further comprising:
providing a touch database, comprising a plurality of breathing condition pattern touches, each of which being divided into a proper breathing pattern touch and an improper breathing pattern touch,
wherein when the processor receives a breathing condition identified as out of a standard range, a touch of an improper breathing condition pattern is retrieved from the touch database and a touch generator of the multi-sensory feedback generator performs a corresponding breathing condition touch, the touch generator being a vibration motor, a thermal source, or a mild electricity source.

27. The method according to claim 19, further comprising:
providing a touch database, comprising a plurality of breathing condition pattern touches, each of which being divided into a proper breathing pattern touch and an improper breathing pattern touch,
wherein when the processor receives a breathing condition identified as within a standard range, a touch of a proper breathing condition pattern is retrieved from the touch database and a touch generator of the multi-sensory feedback generator performs a corresponding breathing condition touch, the touch generator being a vibration motor, a thermal source, or a mild electricity source.

28. The method according to claim 19, further comprising:
providing a hearing database, comprising a plurality of breathing condition voices, each of which being divided into a proper breathing pattern voice and an improper breathing pattern voice,
wherein when the processor receives a breathing condition identified as out of a standard range, a voice of an improper breathing condition pattern is retrieved from the hearing database and a speaker of the multi-sensory feedback generator plays a corresponding breathing condition voice.

29. The method according to claim 19, further comprising:
providing a hearing database, comprising a plurality of breathing condition voices, each of which being divided into a proper breathing pattern voice and an improper breathing pattern voice,
wherein when the processor receives a breathing condition identified as within a standard range, a voice of a proper breathing condition pattern is retrieved from the hearing database and a speaker of the multi-sensory feedback generator plays a corresponding breathing condition voice.

30. The method according to claim 19, further comprising:
providing a vision database, comprising a plurality of breathing condition pictures, each of which being divided into a proper breathing pattern picture and an improper breathing pattern picture,
wherein when the processor receives a breathing condition identified as out of a standard range, a picture of an improper breathing condition pattern is retrieved from the vision database and a monitor of the multi-sensory feedback generator displays a corresponding breathing condition picture.

31. The method according to claim 19, further comprising:
providing a vision database, comprising a plurality of breathing condition patterns, each of which being divided into a proper breathing pattern picture and an improper breathing pattern picture,
wherein when the processor receives a breathing condition identified as out of a standard range, a picture of a proper breathing condition pattern is retrieved from the vision database and a monitor of the multi-sensory feedback generator displays a corresponding breathing condition picture.

32. The method according to claim 30, wherein the breathing condition picture is a static picture or a dynamic picture.

33. The method according to claim 31, wherein the breathing condition picture is a static picture or a dynamic picture.

34. The method according to claim 30, wherein the monitor is a projector, a head mounted display, a PC display, a liquid crystal display, or a screen of a handheld apparatus.

35. The method according to claim 31, wherein the monitor is a projector, a head mounted display, a PC display, a liquid crystal display, or a screen of a handheld apparatus.

36. The method according to claim 19, further comprising:
detecting a physiological data by a physiological detector and providing the physiological data to the processor in facilitating to make more precision judgment according to the breathing condition.
